(19) United States
(12) Patent Application Publication
(10) Pub. No.: US 2009/0275551 A1
(43) Pub. Date: Nov. 5, 2009

(54) Fluvastatin for the treatment of patients with a history of muscle related side effects with other statins

(76) Inventors: Evan A. Stein, Ft. Thomas, KY (US); Claudio Ruben Gimpelewicz, Reinach (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/288,591

(22) Filed: Oct. 22, 2008

Related U.S. Application Data
(60) Provisional application No. 61/001,362, filed on Oct. 31, 2007.

(51) Int. Cl. A61K 31/397; A61K 31/404

(52) U.S. Cl. 514/210.02; 514/419

(57) ABSTRACT

The present disclosure relates to method of using fluvastatin, alone or in combination with ezetimibe, be used to (1) lower cholesterol level, (2) treat hypercholesterolemia, (3) treat mixed dyslipidemia and/or (4) inhibit cholesterol biosynthesis in patients who experienced muscle-related side effects (MRSE). The present disclosure also relates to a pharmaceutical composition comprising ezetimibe and fluvastatin.

### FIELD OF THE DISCLOSURE

This disclosure relates to methods and composition for lowering cholesterol level in patients with a history of muscle related side effect associated with statins.

### BACKGROUND OF THE DISCLOSURE

Hypercholesterolemia and mixed dyslipidemia are important modifiable risk factors of cardiovascular disease, with low density lipoprotein cholesterol (LDL-C) levels constituting the generally accepted primary treatment target. For patients that can not be sufficiently controlled by diet and life style intervention, statins are the most common therapeutic approach for cholesterol/lipid lowering. Statins are also used in combination with other cholesterol-lowering compounds such as ezetimibe. For example, the combination of ezetimibe and simvastatin is marketed as under the trade name Vytorin®.

Although statin treatment is generally well tolerated, it is estimated that 5-10% of patients experience muscle-related side effects (MRSEs), resulting in less effective non-statin alternatives or cessation of lipid-lowering therapy completely. Uncomplicated statin-associated muscle complaints such as myalgia (i.e. muscle pain/weakness without or only with subtle creatine-kinase elevations) are increasingly recognized as an important medical problem reported by approximately 10% of patients in clinical practice with symptoms often occurring during the first few months after treatment initiation.

The absence of effective, reliable pharmacological countermeasures to mitigate statin-associated muscle complaints and the limited LDL-C lowering capacity with alternative lipid lowering treatments such as niacin or bile acid sequestrants or possible tolerability issues with these alternative treatments can cause difficulties in achieving effective long-term lipid lowering in patients who do not tolerate statins.

Temporary discontinuation of statin treatment to allow for disappearance of muscle symptoms followed by a re-challenge with the same statin at the same or a reduced dosage or the switch to an alternative statin are therefore recommended in patients who do not tolerate statins due to muscle complaints. Based on retrospective data, muscle symptom recurrence has however been reported to occur in 50-95% of patients with prior statin-associated muscle symptoms upon re-challenge with the same or a different statin and the rate of premature discontinuations is overall 23% (10/44 patients) in a recent randomized, controlled 12-week study in patients with a history of statin-associated myalgia, who were treated with simvastatin (alone or in combination with co-enzyme Q10).

With an estimated 25 million people worldwide receiving statin therapy, MRSEs in 5-10% of patients resulting in cessation of treatment equates to 1.25-2.5 million patients being denied the most effective class of lipid-lowering agents. This constitutes a major problem in the efforts to reduce cardiovascular disease, especially in patients at high risk. In addition, with recent treatment guidelines advocating more stringent LDL-C targets, there is a clear need for treatment regimens that provide well-tolerated lipid-lowering efficacy. Such targets are generally only achieved with high-dose monotherapy such as 80 mg simvastatin, 40-80 mg of atorvastatin, or 20-40 mg of rosuvastatin. However as shown in the PRIMO study (see Bruckert E, Hayem G, Dejager S, Yau C, Begaud B. Mild to moderate muscular symptoms with high-dosage statin therapy in hyperlipidemic patients—the PRIMO study. Cardiovasc Drugs Ther 2005; 19:403-414), higher doses of statins are associated with increased MRSEs.

Hence, there exists a need for developing new ways to treat hypercholesterolemia and/or mixed dyslipidemia, and lower the cholesterol level in patients with a history of muscle related side effects caused by the use of statins.

### BRIEF DESCRIPTION OF THE FIGURES

FIG. 1 depicts how a total of 251 subjects are screened and 218 patients are randomized to treatment.

FIG. 2 depicts the level of LDL-C level in ezetimibe monotherapy, fluvastatin XL monotherapy and fluvastatin XL/ezetimibe combination therapy.

FIG. 3 depicts the percentage change from baseline in the LDL-C:HDL-C ratio, total cholesterol, triglyceride and Apo-B levels for various therapies.

FIG. 4 depicts the proportion of patients achieved their target LDL-C level on various therapies.

FIG. 5 depicts that in a Kaplan-Meier analysis of time to first MRSE there is no indication for an increased risk of MRSE recurrence with fluvastatin XL alone or in combination with ezetimibe when compared to the non-statin ezetimibe.

### SUMMARY OF THE DISCLOSURE

The present disclosure relates to methods of lowering cholesterol level using fluvastatin in patients who experienced muscle-related side effects.

The present disclosure relates to methods treating hypercholesterolemia using fluvastatin in patients who experienced muscle-related side effects.

The present disclosure relates to methods treating mixed dyslipidemia using fluvastatin in patients who experienced muscle-related side effects.

The present disclosure relates to methods inhibiting cholesterol synthesis using fluvastatin in patients who experienced muscle-related side effects.

The present disclosure relates to methods of lowering cholesterol level using fluvastatin in patients who experienced muscle-related side effects.

The present disclosure relates to methods treating hypercholesterolemia using the combination of fluvastatin and ezetimibe in patients who experienced muscle-related side effects.

The present disclosure relates to methods treating mixed dyslipidemia using the combination of fluvastatin and ezetimibe in patients who experienced muscle-related side effects.

The present disclosure relates to methods inhibiting cholesterol synthesis using the combination of fluvastatin and ezetimibe in patients who experienced muscle-related side effects.

Such muscle-related side effects are caused by other (i.e., non-fluastatin) statins or other lipid lowering treatments.

The present disclosure also relates to a pharmaceutical composition comprising fluvastatin and ezetimibe.

### DETAILED DESCRIPTION OF THE DISCLOSURE

Fluvastatin is a competitive and reversible inhibitor of 3-hydroxy-3-methylglutaryl coenzyme A (HMG-CoA) reductase, the enzyme responsible for the conversion of HMG-CoA to mevalonic acid, a precursor of sterols including cholesterol. One embodiment of the fluvastatin is fluvastatin sodium which has the chemical name: 7-[3-(4-fluorophenyl)-1-(1-methylethyl)-1H-indol-2-yl]-3,5-dihydroxy-hept-6-enoic acid, monosodium salt. Fluvastatin is generally and/or specifically disclosed, for example, by U.S. Pat. Nos. 5,354,772, 5,356,896 and 6,242,003. An extended-release formulation of fluvastatin is fluvastatin XL, or Lescol-XL®.

Ezetimibe (tradename Zetia®) is an anti-hyperlipidemic medication which is used to lower cholesterol levels. It acts by decreasing cholesterol absorption in the intestine. Ezetimibe is used as monotherapy or as combination therapy with HMG-CoA reductase inhibitors as adjunctive therapy to diet for the reduction of elevated TC, LDL-C, and Apo B in patients with primary (heterozygous familial and non-familial) hypercholesterolemia. Ezetimibe is increasingly used in combination with statins for lowering of LDL-C in this indication. Ezetimibe is disclosed, for example, by U.S. Pat. Nos. 5,846,966, 7,030,106 and RE37721.

It has been found that fluvastatin, alone or in combination with ezetimibe, be used to (1) lower cholesterol level, (2) treat hypercholesterolemia, (3) treat mixed dyslipidemia and/or (4) inhibit cholesterol synthesis in patients who experienced muscle-related side effects (MRSE). Such MRSEs include but not limited to myalgia (defined as muscle ache, pain or discomfort), muscle cramps, lack of strength during effort, heaviness and/or weakness without creatine kinase elevation >3 times upper limit of normal). Such MRSE are often associated with prior treatment of non-fluvastatin statins such as atorvastatin, lovastatin, mevastatin, pitavastatin, pravastatin, rosuvastatin, and/or simvastatin. In a particular embodiment, such MRSE are associated with prior treatment of atorvastatin and/or simvastatin.

In one embodiment, the above methods include use of ezetimibe in addition to fluvastatin.

The present disclosure also includes a pharmaceutical composition comprising fluvastatin and ezetimibe.

In a particular embodiment, the fluvastatin in an extended release formulation.

A number of references have been mentioned in this disclosure, all of which are hereby incorporated by reference. If there are any discrepancy between the current disclosure and the references, the current disclosure should be relied on for the purpose of resolving such discrepancy.

### EXAMPLES

### (a) Method

A randomized, double-blind, double-dummy, parallel-group, multicenter study evaluating the efficacy and tolerability of fluvastatin XL 80 mg/day, and ezetimibe 10 mg/day, alone or in combination, is conducted according to the ethical principles of the Guidelines for Good Clinical Practice and the Declaration of Helsinki of the World Medical Association. Patients are recruited in 27 centers in Germany, Greece, Norway, Russia, Turkey and the United States. The protocol is approved by local ethics committees or institutional review boards. Written, informed consent before randomization are provided by all patients.

After a minimum 5-week lead-in, drug wash-out and diet stabilization period, patients who fulfilled the study criteria are randomized in a 1:1:1 ratio to 12-week treatment with fluvastatin XL 80 mg plus matched placebo for ezetimibe 10 mg, or ezetimibe 10 mg plus matched placebo for fluvastatin XL 80 mg, or fluvastatin XL 80 mg plus ezetimibe 10 mg. For blinding purposes tablets for fluvastatin XL/placebo and capsules for ezetimibe/placebo are used. Treatment assignment is unbiased and concealed from patients and investigator staff, by a randomization list produced by the sponsor using a validated system that automates the random assignment of treatment groups. Treatment remains fully blinded from the time of randomization until database lock. Trial medication is taken with water at bedtime. Compliance is assessed by study personnel using pill counts at each visit. Patients are to continue a low saturated fat and cholesterol-restricted diet and exercise regimen.

The study enrolls men and women 18 years of age or older with dyslipidemia who have previously experienced documented MRSEs that have led to cessation of statin treatment, or patients currently receiving statin treatment (other than fluvastatin) whose quality of life is affected by MRSEs and require switching to an alternative treatment. MRSEs include myalgia (defined as muscle ache, pain or discomfort), muscle cramps, lack of strength during effort, heaviness and/or weakness without creatine kinase elevation >3 times upper limit of normal.

Exclusion criteria included: homozygous familial hypercholesterolemia, and Fredrickson Types I, IV and V dyslipoproteinemia; history or evidence of myopathy (muscle pain with creatine kinase elevation >10 times upper limit of normal) or similar asymptomatic creatine kinase elevation; unexplained serum creatine kinase levels >3 times upper limit of normal at the baseline visit; history of rhabdomyolysis, or any congenital muscular disease; history of hypersensitivity to or MRSEs with fluvastatin; history of hypersensitivity to or intolerance of ezetimibe; alanine aminotransferase and/or aspartate aminotransferase >2 times upper limit of normal; severe renal function impairment (defined as creatinine blood level >2.5 mg/dL and/or proteinuria >1.5 g/24 hours); acute coronary syndrome, arterial revascularization, coronary artery bypass graft surgery, and stroke within 6 months before study commencement. Patients receiving drugs metabolized by cytochrome P450 2C9 are also excluded.

Initial screening includes medical history, physical examination, clinical chemistry, hematology and urinalysis. Low-density lipoprotein cholesterol (LDL-C), total and high-density lipoprotein cholesterol (HDL-C), triglycerides, LDL-C:HDL-C ratio, apolipoprotein [Apo] AI and Apo-B are measured at weeks 0, 4, 8 and 12. High sensitivity C-reactive protein (hs-CRP) is measured at weeks 0 and 12. All blood assays, are conducted at a College of American Pathology accredited and Center for Disease Control Part III lipid laboratory. Blood samples are obtained at every visit after at least 10 hours without food or liquid (with the exception of water), non-fasting patients are rescheduled within the following 2-3 days. LDL-C is calculated using the Friedewald formula when triglyceride levels <400 mg/dL. Ultracentrifugation is used when triglyceride levels are >400 mg/dL.

Safety evaluations include regular monitoring of all adverse events, urine, hematology, blood chemistry, as well as physical examination and assessment of vital signs. MRSEs are assessed before and during the study using a standard questionnaire as detailed in Table 1.

TABLE 1
Summary of muscle-related side effect (MRSE) questionnaire

Incidence and time pattern of MRSE
Has the patient reported any muscle-related symptoms as an adverse event? (Yes/No)
Is the pain continuous or intermittent (for minutes, hours or related to effort)? (Multiple choice)
Clinical description of symptoms
Would you describe the pain as weakness, ponderous/heaviness, lack of strength during effort,
muscle ache or cramps? (Multiple choice)
Is the pain diffuse? If no, is it localized to the thigh, calf, arm/forearm, trunk, or is there no
predominate localization? (Multiple choice)
Is there associated tendinous pain? If yes, is it localized to one tendon or are multiple tendons
affected? (Multiple choice)
Would you describe the intensity of discomfort as minor, occurring after light and heavy
activity, occurring after heavy activity only, or major (confinement to bed or incapacity to
work)? (Multiple choice)
Did you experience symptoms of the same kind when you were treated previously with another
statin? (Yes/No)
Triggering factors
Is there one or more identifying circumstance when the pain occurs? If yes, specify whether this
is sport, muscle trauma, cold, unusual physical effort, rest, intake of a new drug or other.
(Multiple choice)
Use of analgesics for muscle pain
Did the pain require symptomatic treatment (analgesics, non-steroidal anti-inflammatory drug or
other)? (Yes/No/Other)
Concomitant medication
Did you take any medications in the 48 hours prior to the start of the episode? (Yes/No) or
other)? (Yes/No/Other)

Objectives of this study are to assess LDL-C reduction from baseline of (1) fluvastatin XL 80 mg in combination with ezetimibe 10 mg compared with ezetimibe 10 mg monotherapy and (2) fluvastatin XL 80 mg compared with ezetimibe 10 mg. Objectives of the study are also include (1) to assess the tolerability of fluvastatin XL 80 mg and ezetimibe 10 mg alone or in combination as determined by the rate of recurrence of MRSEs and the rate of recurrence of MRSEs leading to study drug discontinuation; (2) to assess the effect of fluvastatin XL 80 mg alone or in combination with ezetimibe 10 mg on total cholesterol, triglycerides, HDL-C, LDL-C:HDL-C ratio, Apo-AI and Apo-B compared with ezetimibe 10 mg monotherapy; (3) to determine the number of patients in each treatment group who reached target LDL-C levels according to the current National Cholesterol Education Program guidelines, (4) to assess the effect of fluvastatin XL 80 mg and ezetimibe 10 mg alone or in combination on hs-CRP; and (5) to assess the overall safety and tolerability profile of fluvastatin XL 80 mg and ezetimibe 10 mg alone or in combination.

The study would be able to show that fluvastatin XL 80 mg is superior to ezetimibe 10 mg in reducing LDL-C. With a sample size of 70 patients per treatment group, the study has 90% power to detect a difference of 9.65% between the population means, using a two-sided comparison with α equal to 0.05 and assuming a standard deviation of 17.5% in both groups. Efficacy and tolerability analyses are performed using the intent-to-treat population, defined as all patients receiving at least 1 dose of study drug. The safety population includes all patients receiving at least 1 dose of study drug and had at least 1 post-baseline safety assessment.

The primary endpoint is the percentage reduction in LDL-C from baseline to week 12 (or study end using last observation carried forward). For the analysis, 2 primary hypotheses are tested using a hierarchical testing procedure, i.e, if the comparison between the combination of fluvastatin XL/ezetimibe and ezetimibe alone is statistically significant (p<0.05), the percentage change in LDL-C achieved with fluvastatin XL alone is compared with ezetimibe alone in a second step. Changes in LDL-C are compared using an ANCOVA (analysis of covariance) model, with treatment group and pooled center (pooled within country) as factors and baseline LDL-C as a covariate. Two-sided 95% confidence intervals (CI) and the corresponding p-values are calculated for between-treatment differences. An analogous ANCOVA model is used for the analysis of continuous secondary variables. Triglycerides, LDL-C:HDL-C ratio, HDL-C, Apo-AI and Apo-B are log-transformed prior to analysis. Least squares mean estimates are calculated to express percentage reductions from baseline. Comparisons between treatment groups are presented both as additive and percentage comparisons. Mean differences and the ratio of the treatment group means along with the 95% CI and p-values from the pair-wise comparisons are calculated. The proportion of patients achieving LDL-C goal are compared for each pair of treatment groups and the Cochran-Mantel-Haenszel test stratified by center is used to assess overall treatment difference. Odds ratios and 95% CI are calculated. The percentage change for hs-CRP is analyzed using a Wilcoxon sum rank test. The difference between treatment medians and the corresponding 95% CI are based on the Hodges-Lehmann estimate. Time to recurrence of MRSEs from start of treatment is assessed using a proportional hazards model with treatment and time since previous statin treatment (<6 months, 6 months-1 year, >1 year) as factors. The hazard ratio, 95% CI and corresponding p-values for the time to recurrence are derived from the model and corresponding Kaplan-Meier curves are plotted. The numbers and percentages of patients experiencing a recurrence of MRSEs are assessed by treatment group using a logistic regression model with factors for treatment group, pooled center and previous statin treatment. The odds ratio, 95% CI and corresponding p-values for the incidence of recurrence are derived from the model. Analyses are repeated for MRSEs leading to study discontinuation.

This is an active-controlled study and it is therefore unknown whether a similar incidence of MRSEs would have occurred in a placebo-treated group. The use of placebo in what is generally a moderate and high CVD risk, hypercholesterolemic population is considered inappropriate and the selection of the non-statin ezetimibe is determined to be an acceptable alternative. Ezetimibe has a very low systemic exposure, and is not known to enter skeletal muscle in any significant amount or interfere with the cholesterol synthesis pathway, although myalgia with monotherapy has recently reported in a patient with a defect in fatty acid oxidation.

### (b) Results

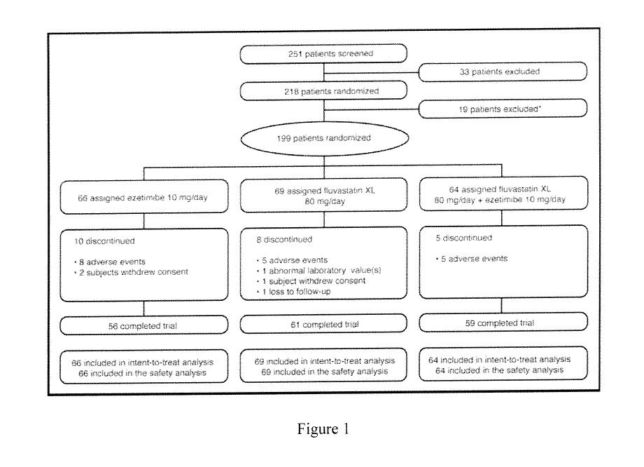

A total of 251 subjects are screened and 218 patients are randomized to treatment (FIG. 1). An audit by the sponsor of one of the study centers detects significant irregularities related to another study, however, there are no such findings related to this trial. As a precautionary measure, patients recruited at this center (n=19) are excluded from the final analyses, leaving 199 patients in the intent to treat/safety population. Inclusion or exclusion of these 19 patients does not affect the overall conclusions of the study. A total of 176 (88%) patients have completed the study, reasons for discontinuation are shown in FIG. 1.

Baseline characteristics for age, sex, race and body mass index (BMI) are comparable in all 3 treatment groups (Table 2). Approximately 46% of patients are in the National Cholesterol Education Program Adult Treatment Program III high-risk category. Baseline levels of lipids and lipoproteins are similar for the 3 treatment groups, as is previous use of statins, history of MRSEs (with and without statin use) and asymptomatic creatine kinase elevations. Prior statin use is shown in (Table 3). Patients with a history of MRSEs have most commonly received simvastatin or atorvastatin therapy (127 [64%] and 67 [34%], respectively).

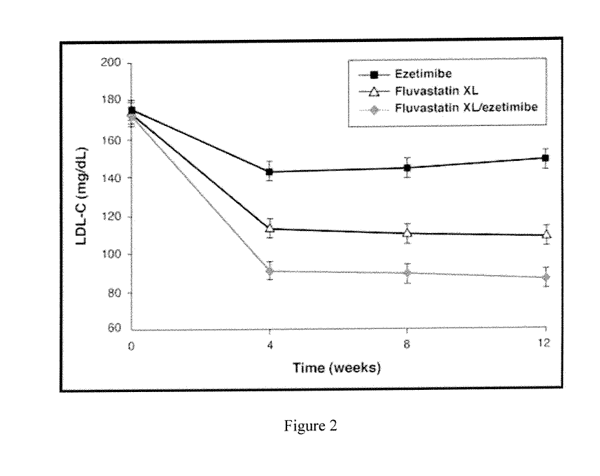

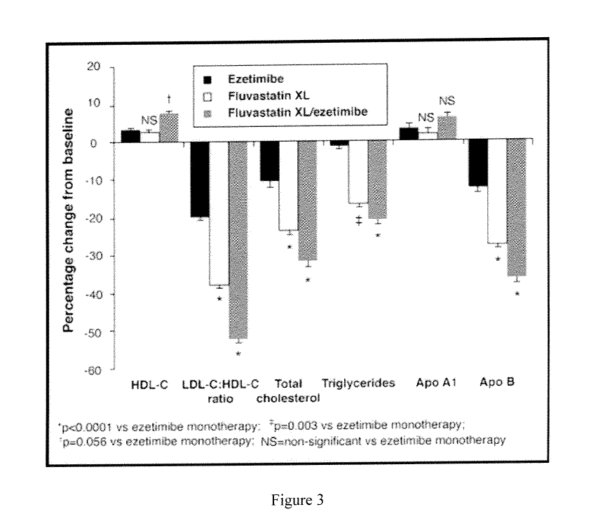

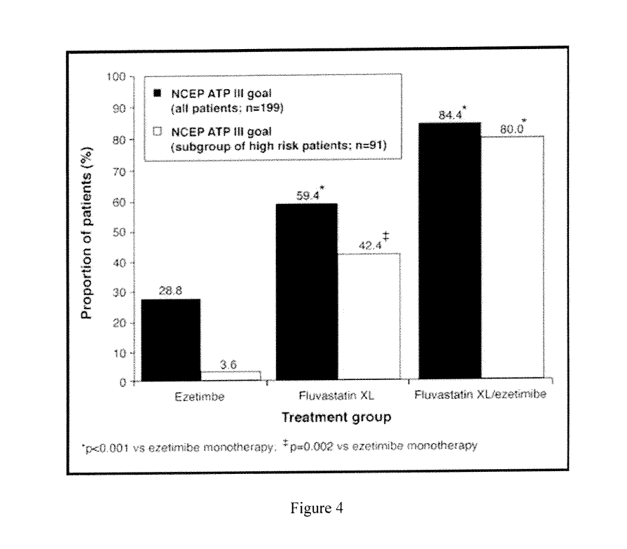

Ezetimibe monotherapy, fluvastatin XL monotherapy and fluvastatin XL/ezetimibe combination therapy lowered LDL-C levels by 15.6%, 32.8% and 46.1%, respectively (between-group difference: fluvastatin XL vs ezetimibe −17.1%, 95% CI-23.6 to −10.7%; p<0.0001; combination vs ezetimibe −30.4%, 95% CI-37.0 to −23.8%; p<0.0001) (FIG. 2). Fluvastatin XL alone or in combination with ezetimibe provides significantly greater reductions in the LDL-C:HDL-C ratio, total cholesterol, triglyceride and Apo-B levels compared with ezetimibe monotherapy (all p<0.0001, with the exception of triglyceride for fluvastatin XL vs ezetimibe, p=0.003) (FIG. 3). More patients on fluvastatin XL monotherapy and fluvastatin XL/ezetimibe combination therapy achieved their target LDL-C level compared with ezetimibe monotherapy (p<0.001 for fluvastatin XL monotherapy or combination therapy vs ezetimibe monotherapy; FIG. 4). In the subgroup of 91 patients that are in the high-risk category, 42% of patients receiving fluvastatin XL monotherapy and 80% on fluvastatin XL/ezetimibe combination therapy achieve a LDL-C level of <100 mg/dL at study end compared with 4% receiving ezetimibe monotherapy (p<0.001 for fluvastatin XL/ezetimibe combination therapy vs ezetimibe monotherapy and p=0.002 for fluvastatin XL monotherapy vs ezetimibe monotherapy) (FIG. 4). Median hs-CRP levels are reduced by 18.6% from baseline with fluvastatin XL/ezetimibe combination therapy which is significantly greater than the 0% change achieved with ezetimibe monotherapy (p=0.04). The reduction of 7.9% for fluvastatin XL monotherapy is not significantly different to ezetimibe monotherapy (p=0.53).

There are no treatment related serious adverse events, no rhabdomyolysis or creatine kinase elevations ≧10 times upper limit of normal. Overall, 107 patients (54%) report an adverse event (39 patients on ezetimibe, 34 patients on fluvastatin XL and 34 patients on fluvastatin XL/ezetimibe) and 18 patients (9%) prematurely discontinue from the study due to adverse events (8 patients on ezetimibe, 5 patients on fluvastatin XL and 5 patients on fluvastatin XL/ezetimibe).

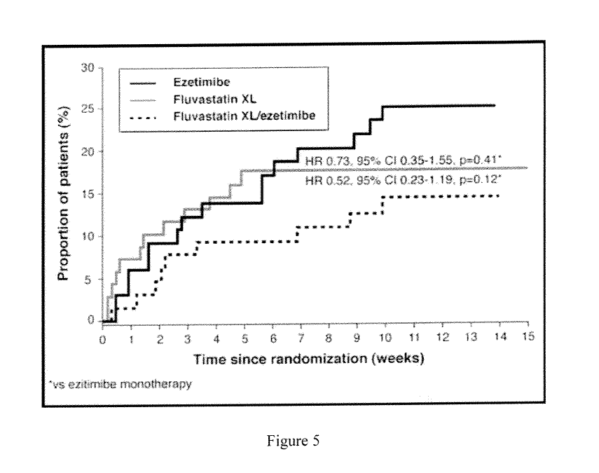

MRSEs are the most frequent type of adverse events, overall reported in 37 patients (19%) and of mild to moderate intensity in most cases. In the ezetimibe group 16 patients (24%) reported a MRSE compared with 12 patients (17%) in the fluvastatin XL group and 9 patients (14%) in the fluvastatin XL/ezetimibe combination group. MRSEs led to study discontinuation in 5 patients (8%) on ezetimibe, in 3 patients (4%) on fluvastatin XL and in 2 patients (3%) on fluvastatin XL/ezetimibe (Table 4). In a Kaplan-Meier analysis of time to first MRSE there is no indication for an increased risk of MRSE recurrence with fluvastatin XL (FIG. 5). Differences in the recurrence of MRSEs are not statistically different between the treatment groups, but tend to be lower in patients on fluvastatin XL/ezetimibe combination therapy (hazard ratio 0.52, 95% CI-0.23-1.19) compared with patients receiving ezetimibe monotherapy. An asymptomatic creatine kinase elevation between ≧5 and <10 times upper limit of normal is reported in 1 patient on a single occasion, which normalizes while continuing study drug treatment. Consecutive aspartate aminotransferase and/or alanine aminotransferase elevations >3 times upper limit of normal are observed in 1 patient each on fluvastatin XL/ezetimibe combination treatment and on fluvastatin XL monotherapy. Neither of these patients have clinical symptoms or other liver function test abnormalities. Transaminase levels return to the normal range within 1-2 weeks of the last study drug intake.

TABLE 2
Baseline and demographic characteristics.

| | | | Fluvastatin |
|---|---|---|---|
| | Ezetimibe | Fluvastatin XL | XL/ezetimibe |
| | (n = 66) | (n = 69) | (n = 64) |
| Age (years) | 61.4 ± 10.1 | 60.6 ± 9.7 | 61.0 ± 10.5 |
| Men | 28 (42%) | 36 (52%) | 31 (48%) |
| Caucasian | 65 (98%) | 69 (100%) | 63 (98%) |
| Body Mass Index (kg/m²) | 28.4 ± 4.8 | 28.2 ± 4.2 | 29.2 ± 4.4 |
| Classification of risk | | | |
| High | 28 (42%) | 33 (48%) | 30 (47%) |
| Moderate | 15 (23%) | 22 (32%) | 17 (27%) |
| Low | 23 (35%) | 14 (20%) | 17 (27%) |
| LDL-C (mg/dL) | 176.2 ± 40.0 | 174.2 ± 48.0 | 172.9 ± 44.1 |
| HDL-C (mg/dL) | 52.4 ± 15.1 | 53.6 ± 14.4 | 55.2 ± 15.3 |
| LDL-C:HDL-C ratio | 3.6 ± 1.0 | 3.4 ± 1.3 | 3.3 ± 1.0 |
| Total cholesterol (mg/dL) | 268.7 ± 43.7 | 262.9 ± 53.6 | 265.6 ± 47.2 |
| Triglycerides (mg/dL) | 197.0 ± 104.6 | 175.5 ± 82.4 | 188.4 ± 100.9 |
| Median | 162.4 | 164.6 | 168.6 |
| Apolipoprotein AI (mg/dL) | 166.0 ± 30.2 | 168.6 ± 29.3 | 169.4 ± 29.1 |
| Apolipoprotein B (mg/dL) | 168.3 ± 31.3 | 162.0 ± 38.0 | 160.9 ± 30.7 |
| High sensitivity C Reactive | 1.8 | 1.7 | 2.5 |
| Protein Median (mg/L) | | | |

Data are presented as mean ± SD unless otherwise stated.
Classification of risk is based on National Cholesterol Education Program Adult Treatment Panel III guidelines.
HDL-C = high-density lipoprotein cholesterol; LDL-C = low-density lipoprotein cholesterol.

TABLE 3
Previous statin usage and history of muscular symptoms at screening

| | | | |
|---|---|---|---|
| | | Fluvastatin | |
| Ezetimibe | Fluvastatin XL | XL/ezetimibe | Total |
| (n = 66) | (n = 69) | (n = 64) | (n = 199) |

Previous statin use

| | | | | |
|---|---|---|---|---|
| <6 months | 44 (67%) | 47 (68%) | 47 (73%) | 138 (69%) |
| 6 to <12 | 8 (12%) | 6 (9%) | 10 (16%) | 24 (12%) |
| months | | | | |
| >12 months | 14 (21%) | 16 (23%) | 7 (11%) | 37 (19%) |

History of muscle related side effects

| | | | | |
|---|---|---|---|---|
| Yes* | 66 (100) | 69 (100%) | 64 (100%) | 199 (100%) |
| Simvastatin | 40 (61%) | 50 (73%) | 37 (58%) | 127 (64%) |
| Pravastatin | 8 (12%) | 4 (6%) | 10 (16%) | 22 (11%) |
| Atorvastatin | 25 (38%) | 20 (29%) | 22 (34%) | 67 (34%) |
| Rosuvastatin | 2 (3%) | 6 (9%) | 3 (5%) | 11 (6%) |
| Lovastatin | 2 (3%) | 1 (1%) | 2 (3%) | 5 (3%) |
| Cerivastatin | 0 | 2 (3%) | 1 (2%) | 3 (2%) |

History of asymptomatic elevation of creatine kinase

| | | | | |
|---|---|---|---|---|
| No | 62 (94%) | 62 (90%) | 58 (91%) | 182 (92%) |
| Yes | 4 (6%) | 7 (10%) | 6 (9%) | 17 (8%) |

History of muscular symptoms without medication

| | | | | |
|---|---|---|---|---|
| No | 61 (92%) | 67 (97%) | 59 (92%) | 187 (94%) |
| Yes | 5 (8%) | 2 (3%) | 5 (8%) | 12 (6%) |

Family history of muscular symptoms with statins

| | | | | |
|---|---|---|---|---|
| No | 37 (56%) | 42 (61%) | 43 (67%) | 122 (61%) |
| Yes | 0 | 1 (1%) | 1 (2%) | 2 (1%) |
| Unknown | 29 (44%) | 26 (38%) | 20 (31%) | 75 (38%) |

The numbers in each column may not add up to the total numbers of patients because patients may have received more than one statin.

TABLE 4
Incidence of and time to first muscle-related side effect

| | | Number of | |
|---|---|---|---|
| | | patients | Median time |
| | | (incidence | to first |
| | Treatment Group | rate) | event (weeks) |
| Any MRSE | Ezetimibe (n = 66) | 16 (24%) | 3.07 |
| recurrence | Fluvastatin XL (n = 69) | 12 (17%) | 1.36 |
| | Fluvastatin XL/ezetimibe | 9 (14%) | 2.14 |
| | (n = 64) | | |
| MRSE | Ezetimibe (n = 66) | 5 (8%) | 2.57 |
| recurrence | Fluvastatin XL (n = 69) | 3 (4%) | 0.43 |
| leading to | Fluvastatin XL/ezetimibe | 2 (3%) | 1.57 |
| discontinuation | (n = 64) | | |

MRSE = muscle-related side effect

### (c) Discussion

The present trial demonstrates that most patients who are intolerant to other statins, due to muscular complaints without creatine kinase elevations, can tolerate fluvastatin XL 80 mg/day either as monotherapy or in combination with ezetimibe 10 mg/day. Fluvastatin XL 80 mg/day administered in combination with ezetimibe 10 mg/day provides substantial additional lowering of LDL-C levels compared with the respective monotherapies (additional 13.3% reduction vs fluvastatin XL and additional 30.4% reduction vs ezetimibe), an additive effect similar to that achieved when ezetimibe is combined with other statins. The lack of reduction in hs-CRP with ezetimibe monotherapy is consistent with previous reports, while the 18% reduction achieved with the combination of fluvastatin XL and ezetimibe is consistent with the additional decrease in hs-CRP seen when ezetimibe is added to other statins. Even though the study population comprised patients with a history of MRSEs with prior statin therapy, approximately 85% of patients do not experience a MRSE when treated with fluvastatin XL either alone or in combination with ezetimibe, and only 3-4% are discontinued from the 12-week study treatment because of MRSEs.

The 12-week duration of this trial, while relatively short in terms of expected duration of therapy in clinical practice, is consistent with the PRIMO study, which showed 75% of patients who reported MRSEs did so within the first 0.3 months of starting statin therapy. The fairly rapid onset of MRSEs with statins is confirmed in this trial, especially with fluvastatin XL where it reaches a plateau at 5 weeks (FIG. 5). A high frequency of adverse events for musculoskeletal and connective tissue disorders in this study is to be expected, given that trial participants are selected for a history of statin-associated MRSEs.

The LDL-C reductions achieved in this study with combination therapy are comparable to those reported with these high-dose statin therapies ((1) Jones P H, Davidson M H, Stein E A, Bays H E, McKenney J M, Miller E, Cain V A, Blasetto J W; STELLAR Study Group. Comparison of efficacy and safety of rosuvastatin versus atorvastatin, simvastatin, and pravastatin across doses (STELLAR Trial). Am J Cardiol 2003; 92:152-160.; (2) Knopp RH. Drug treatment of lipid disorders. N Engl J Med 1999; 341: 498-511), or combination of lower doses in combination with ezetimibe (Ballantyne C M, Abate N, Yuan Z, King T R, Palmisano J. Dose-comparison study of the combination of Ezetimibe and simvastatin (Vytorin) versus atorvastatin in patients with hypercholesterolemia: The Vytorin Versus Atorvastatin (VYVA) Study. Am J Heart 2005; 149:464-473)). Moreover, in this study there is no increase in the proportion of patients experiencing MRSEs during combination therapy compared to respective monotherapies.

Without being bound by any particular theory, this tolerability profile of fluvastatin XL may be attributable to its unique pharmacokinetic properties; the slow release of fluvastatin XL from the gastrointestinal tract increases first-pass hepatic uptake, avoiding hepatic saturation and thereby markedly reducing peripheral blood concentrations of the drug may contribute to a slower and lower rate of passage into muscle cells, which is dependent on passive diffusion.

What is claimed is:

1. A method for lowering cholesterol level comprising administering fluvastatin to patients who experienced muscle-related side effect(s).

2. The method of claim 1 wherein the muscle-related side effect(s) is associated with prior use of a statin other than fluvastatin.

3. The method of claim 2, wherein the muscle-related side effect(s) is associated with prior use of atorvastatin and/or simvastatin.

4. The method of claim 1, wherein ezetimibe is used in addition to fluvastatin.

5. The method of claim 1, wherein the fluvastatin is an extended release formulation.

6. The method of claim 1, wherein the cholesterol is low density lipoprotein cholesterol (LDL-C).

7. A method for treating hypercholesterolemia comprising administering fluvastatin to patients who experienced muscle-related side effect(s).

8. The method of claim 7 wherein the muscle-related side effect(s) is associated with prior use of a statin other than fluvastatin.

9. The method of claim 8, wherein the muscle-related side effect(s) is associated with prior use of atorvastatin and/or simvastatin.

10. The method of claim 7, wherein ezetimibe is used in addition to fluvastatin.

11. The method of claim 7, wherein the fluvastatin is an extended release formulation.

12. A method for treating mixed dyslipidemia comprising administering fluvastatin to patients who experienced muscle-related side effect(s).

13. The method of claim 12 wherein the muscle-related side effect(s) is associated with prior use of a statin other than fluvastatin.

14. The method of claim 13, wherein the muscle-related side effect(s) is associated with prior use of atorvastatin and/or simvastatin.

15. The method of claim 12, wherein ezetimibe is used in addition to fluvastatin.

16. The method of claim 12, wherein the fluvastatin is an extended release formulation.

17. A method for inhibiting cholesterol biosynthesis comprising administering fluvastatin to patients who experienced muscle-related side effect(s).

18. The method of claim 17 wherein the muscle-related side effect(s) is associated with prior use of a statin other than fluvastatin.

19. The method of claim 18, wherein the muscle-related side effect(s) is associated with prior use of atorvastatin and/or simvastatin.

20. The method of claim 17, wherein ezetimibe is used in addition to fluvastatin.

21. The method of claim 17, wherein the fluvastatin is an extended release formulation.

22. The method of claim 17, wherein the cholesterol is low density lipoprotein cholesterol (LDL-C).

23. A pharmaceutical composition comprising ezetimibe and fluvastatin.

24. The composition of claim 23, wherein the fluvastatin is an extended release formulation.
